(19) United States
(12) Patent Application Publication
(10) Pub. No.: US 2022/0160988 A1
(43) Pub. Date: May 26, 2022

(54) Respiratory Support Device and Method of Providing Hypoxemia Relief

(71) Applicant: BALTIMORE RESPIRATORY INNOVATIONS LLC, Baltimore, MD (US)

(72) Inventors: Jonathan Thomas Smith, Baltimore, MD (US); Ana Ainechi, Roslyn Heights, NY (US); Brice Dudley, Jamaica Plain, MA (US); Jessica Dakkak, Baltimore, MD (US); Moriah Mattson, Marietta, GA (US); Wilson Tang, Baltimore, MD (US)

(73) Assignee: BALTIMORE RESPIRATORY INNOVATIONS LLC, Baltimore, MD (US)

(21) Appl. No.: 17/442,341

(22) PCT Filed: Mar. 9, 2020
(86) PCT No.: PCT/US20/21706

§ 371 Date: Sep. 23, 2021

Related U.S. Application Data
(60) Provisional application No. 62/815,210, filed on Mar. 7, 2019.

(51) Int. Cl. A61M 16/12; A61M 16/00

(52) U.S. Cl. CPC A61M 16/12; A61M 2202/0208; A61M 16/0069; A61M 16/0063

(57) ABSTRACT

Provided herein is a method of ambulatory respiratory support where LTOT by concentrated oxygen and high flow air delivery are used to create a new form of respiratory support wherein the benefits of both methods can be achieved. This is delivered via a respiratory circuit in combinations of intermittent and continuous flow modalities.

### FIELD

Described herein is an oxygen supplementation device, and a method of delivering oxygen to a subject in need of such treatment.

### BACKGROUND

In the United States, there are currently an estimated 2.4 million individuals who require Long Term Oxygen Therapy (LTOT) to treat hypoxemia (low blood oxygenation) and dyspnea (shortness of breath). These symptoms are commonly associated with chronic respiratory diseases including but not limited to Chronic Obstructive Pulmonary Disease (COPD), Interstitial Lung Disease (ILD), and Pulmonary Hypertension (PH). LTOT treats hypoxemia and dyspnea by providing users with supplemental oxygen to increase the concentration of oxygen users are breathing, increasing their blood oxygenation level (SpO₂). Most patients require <6LPM to maintain a healthy SpO₂ level; however, patients can require >6LPM with the progression of their disease.

LTOT users are prescribed a flow rate of oxygen based on their SpO₂ reading during a walking or exercise-based test, but often users are recommended to increase their oxygen with higher exertion. Individuals may be prescribed oxygen up to 24 hours a day, only when needed, or only with ambulation. Inside the home and in non-portable settings, LTOT users tend to use a stationary concentrator, which requires constant power and can provide a wide range of flows. Outside the home, people most commonly use portable oxygen concentrators (POCs) or oxygen tanks (high-pressure oxygen cylinders). Both options have their limitations: POCs are limited in the flow rates and battery life they provide, while oxygen tanks are heavy and contain a finite volume of oxygen. Both portable options can administer either continuous flow oxygen or pulsed flow oxygen (triggered with inhalation). Pulsed flow conserves oxygen by only administering oxygen when a breath is detected by pressure sensing through a respiratory circuit. Continuous flow has been shown to treat hypoxemia and dyspnea more effectively than pulsed flow, in addition to being more comfortable for oxygen users, but is not practical with higher flow rates of oxygen.

High flows (up to 60LPM) of respiratory gases delivered via nasal cannula (known as nasal insufflation) assist in respiration through several distinct mechanisms. The washout of the non-perfusing sections of the lung and upper airways (called the respiratory dead space) increases breathing efficiency and alveolar FiO₂. The addition of high flow air delivery can also decrease the amount of oxygen required to effectively treat hypoxemia. With high flow air added to an oxygen supplementation device, there is potential to reduce power consumption and increase both battery life and therapeutic efficacy of LTOT.

### SUMMARY

Provided herein is source of oxygen supplementation that supplies oxygen at a flow rate required by the LTOT user. This oxygen delivery can occur in a variety of different flow modalities. As part of some flow modalities, high flow air will be delivered to the LTOT user. As part of some flow modalities, the oxygen required will be provided on a real time basis determined by a change in pressure in the cannula triggered by inhalation pressure from the nasal passages of the oxygen user.

Also described herein is a method of respiratory support that provides oxygen supplementation as well as the benefits of high flow air delivered through a multi-lumen nasal cannula. The system and method described herein allows respiratory support through multiple flow modalities that are combinations of high flow air and oxygen delivered continuously or intermittently. The air source will allow air flow to the respiratory circuit at a flow rate between 10 LPM and 60 LPM. The oxygen source will provide oxygen flow rates between 0.1 LPM and 15 LPM with a purity between 80-96% to the respiratory circuit. The combination of these two methods will reduce the work of breathing to the LTOT user as well as reduce the oxygen required to treat patients.

### BRIEF DESCRIPTION OF THE DRAWINGS

FIG. 1 is a graph depicting a flow modality where pulsed high flow air and oxygen are delivered out-of-phase.

FIG. 2 is a graph depicting a flow modality where pulsed high flow air is delivered with continuous oxygen.

FIG. 3 is a graph depicting a flow modality where continuous high flow air is delivered with pulsed oxygen.

FIG. 4 is a graph depicting a flow modality where high flow air and oxygen are delivered continuously.

FIG. 5 is a diagram illustrating a system where the oxygen source and air source originate from the same source of compressed air.

FIG. 6 is a diagram illustrating a system where the oxygen source and air source originate from different sources of compressed air.

FIG. 7 is a diagram illustrating a system where the oxygen source is compressed oxygen and the air source is compressed air.

### DETAILED DESCRIPTION

Described herein is a system in which flows of air and concentrated oxygen are delivered to the nasal passageway of a patient via a multi-lumen respiratory circuit. In an alternative embodiment of this system the oxygen flow is delivered only during inspiration. The use of a multi-lumen circuit will allow for maintenance of oxygen purity and prevent dilution by ambient air. As used herein, the terms “patient,” “user,” and “subject” are interchangeable and will typically refer to a human or other organism in need of treatment for hypoxia and/or dyspnea.

It is understood that providing air flows around 20LPM assists in the reduction of work of breathing through respiratory support mechanisms such as dead space washout. It is also understood that the provision of air flows greater than 15LPM can assist in the reduction of oxygen required for LTOT treatment. These insights are combined herein to create a new form of respiratory support which simultaneously achieves the benefits of oxygen therapy and high flow air delivery. This system may advantageously be made portable to provide the ambulatory patient with oxygen therapy.

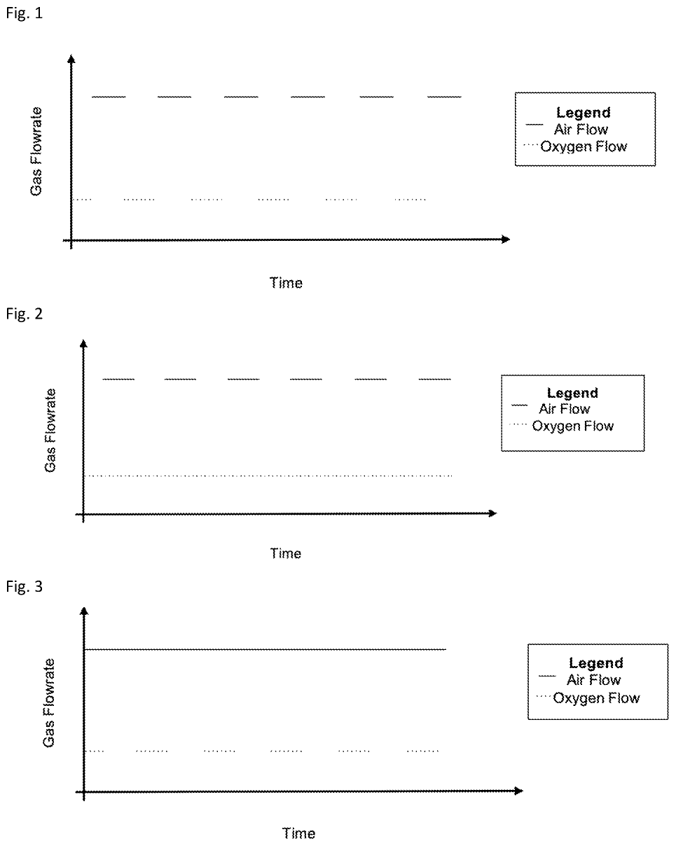

FIG. 1 details an example of a first alternative flow modality that provides the effect detailed above. Both the air and oxygen flows are asynchronous such that the flows are at least partially out of phase in pulse waveforms. The air flow rate in this example will be at a higher rate than the oxygen flow rate. The length of a pulse of both streams is determined by the user's respiratory rate such that there is sufficient time for respiratory dead space washout, determined by the user's respiratory rate. The air flow is synchronized to the user's expiratory period while the oxygen flow is synchronized to the user's inspiratory period.

FIG. 2 details an example of a second alternative flow modality that provides the effect detailed above. The air flow is intermittent while the oxygen flow is continuous. The air flow will be at a higher flow rate than the oxygen flow rate. The length of a pulse in the air flow stream is determined by the user's respiratory rate such that there is sufficient time for respiratory dead space washout, determined by a patient's respiratory rate. The air flow is synchronized to the user's expiratory period.

FIG. 3 details an example of a third alternative flow modality that provides the effect detailed above. The oxygen flow is intermittent while the air flow is continuous. The continuous air flow will be at a higher flow rate than the oxygen flow rate. The length of a pulse in the oxygen flow stream is determined by the user's respiratory rate such that the oxygen pulse is synchronized to the user's inspiratory period.

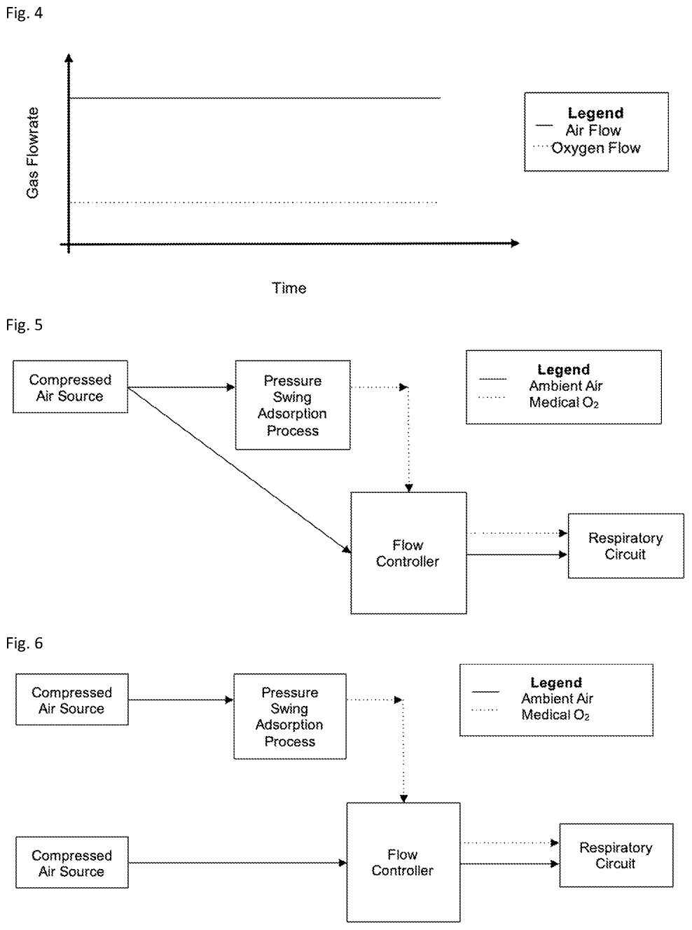

FIG. 4 details an example of a fourth alternative flow modality that provides the effect detailed above. Both the air and oxygen flows are continuous. The continuous air flow will be at a higher flow rate than the oxygen flow rate.

FIG. 5 details an example of a first alternative system which provides flow modalities such as those detailed in FIG. 1-4. An air source, which commonly could be either a tank of liquid or compressed air or an air compressor that compresses the surrounding ambient air, will provide air gas both directly to the flow controller and to the Pressure Swing Adsorption (PSA) unit, which will output oxygen to the flow controller. The flow controller would control these as inputs into the respiratory circuit to provide the previously mentioned flow modalities.

FIG. 6 details an example of a second alternative system that provides the flow modalities such as those detailed in FIG. 1-4. An air source, which commonly could be either a tank of liquid or compressed air or an air compressor that compresses the ambient air around it, will provide air gas to the PSA unit, which will output oxygen to the flow controller. A separate compressed air source will provide air to the flow controller. The flow controller would control these as inputs into the respiratory circuit to provide the previously mentioned flow modalities.

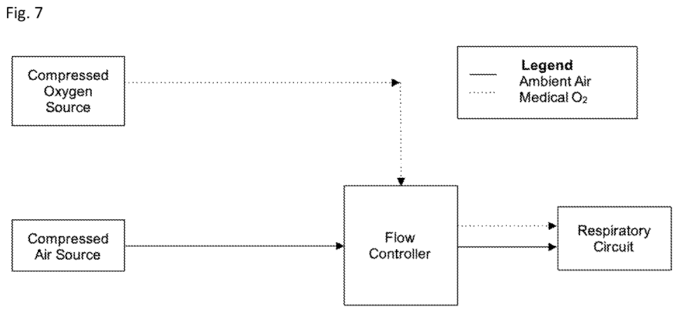

FIG. 7 details an example of a third alternative system that can provide the flow modalities such as those detailed in FIG. 1-4. Any source of air, such as, for example, a tank of liquid compressed or compressed air, or an air compressor that compresses the ambient air around it, will provide air gas to the flow controller. A separate oxygen source, which may be, for example, a tank of liquid or compressed oxygen, will provide oxygen to the flow controller. The flow controller controls these sources as inputs into the respiratory circuit to provide the previously mentioned flow modalities.

Also provided herein is a method of providing oxygen supplementation to a patient suffering from hypoxemia and/or dyspnea, the method comprising delivering to the patient a flow of room air and a separate flow of oxygen. In this method, the flow of oxygen is between about 0.1 LPM and about 15 LPM, such as about 0.05 LPM, about 0.06 LPM, about 0.07 LPM, about 0.08 LPM, about 0.09 LPM, about 0.10 LPM, about 0.2 LPM, about 0.3 LPM, about 0.4 LPM, about 0.5 LPM, about 0.6 LPM, about 0.7 LPM, about 1 LPM, about 2 LPM, about 3 LPM, about 4 LPM, about 5 LPM, about 6 LPM, about 7 LPM, about 8 LPM, about 9 LPM, about 10 LPM, about 11 LPM, about 12 LPM, about 13 LPM, about 14 LPM, or about 15 LPM. The flow of room air is between about 10 LPM and about 60 LPM, for example, about 10 LPM, about 11 LPM, about 12 LPM, about 13 LPM, about 14 LPM, about 15 LPM, about 16 LPM, about 17 LPM, about 18 LPM, about 19 LPM, about 20 LPM, about 25 LPM, about 30 LPM, about 35 LPM, about 40 LPM, about 45 LPM, about 50 LPM, about 55 LPM, or about 60 LPM. In the context of the methods and devices described herein, the term “about” in connection with a parameter, such as flow rate, will be understood to mean that the parameter meets the stated numerical limitation within the limits of normal laboratory testing equipment that is standard in the relevant art, such that the term encompasses some minimal expected amount of variation around the stated numerical limitation.

For use in the method described herein, the flow of oxygen will comprise oxygen in a concentration which is typical of readily available oxygen sources, such as portable oxygen tanks or hospital oxygen supplies. For example, the flow of oxygen may comprise oxygen in a concentration of greater than about 60%, greater than about 70%, greater than about 80%, or greater than about 90%. In one embodiment, the flow of oxygen will comprise oxygen in a concentration of greater than about 87%.

In the method described herein, separate flows of room air and oxygen are delivered simultaneously to the patient. In one embodiment, the separate flows of room air and oxygen are delivered via a multi-lumen cannula. In one embodiment, a multi-lumen cannula is configured so as to prevent mixing of oxygen and room air prior to any mixing in the patient's airway, thereby avoiding dilution of the oxygen with room air prior to delivery to the patient.

The separate flows of oxygen, the room air, or both may be delivered in a pulsatile manner. In one embodiment, the oxygen is delivered continuously, and the room air is delivered in a pulsatile manner. In an alternative embodiment, the oxygen is delivered in a pulsatile manner, and the room air is delivered continuously. In another alternative embodiment, both the oxygen and the room air are delivered in a pulsatile manner. Finally, both the oxygen and the room air may be delivered continuously.

When the oxygen and/or the room air are delivered in a pulsatile manner, the pulses may be synchronized to the patient's breathing. When both streams are pulsed, both streams may be synchronized to the patient's breathing; alternatively, the pulses of oxygen and room air may be delivered at least partially out of phase. In order to synchronize the pulses of oxygen and/or room air to the patient's breathing, the method may include the additional step of measuring the patient's respiratory rate, and delivering the oxygen and/or room air in pulses synchronized to the patient's respiratory rate. The rate of pulses of oxygen and/or room air may vary from about 10 per minute to about 50 per minute.

The volume and duration of the pulses of oxygen and/or room air may be variable, and may be adjusted by the patient or another person, such as a health care professional. Generally, the pulse volume will vary from about 15 to about 200 ml, and the pulse duration will vary from about 0.3 seconds per pulse to about 3 seconds per pulse. In one embodiment, the duration of the pulses will be about 0.3 seconds per pulse, or about 0.6 seconds per pulse, or about 0.9 seconds per pulse, or about 1.2 seconds per pulse, or about 1.5 seconds per pulse, or about 1.8 seconds per pulse, or about 2.1 seconds per pulse, or about 2.4 seconds per pulse, or about 2.7 seconds per pulse, or about 3 seconds per pulse. When synchronized to patient's breathing, pulses of oxygen or room air which will typically have a duration of about 0.25 to about 0.7 of a respiratory cycle, wherein the respiratory cycle is one inhalation and one exhalation.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. It will be appreciated that the spirit and scope of this invention is not limited to the selected examples and embodiments, and that the scope of this invention is defined separately in the appended claims. It will also be appreciated that the figures are not drawn to any particular proportion or scale, and that many variations can be made to any particular proportion or scale, and that many variations can be made to the illustrated embodiments without departing from the spirit of this invention.

1. A method of providing oxygen supplementation to a patient suffering from hypoxemia and/or dyspnea, the method comprising delivering to the patient a flow of room air and a separate flow of oxygen, where the flow of oxygen is between about 0.1 LPM and about 15 LPM, and the flow of room air is between about 10 LPM and about 60 LPM.

2. The method of claim 1, wherein the flow of oxygen comprises oxygen in a concentration of about 80% or greater.

3. The method of claim 2, wherein the concentration of the oxygen is about 87% or greater.

4. The method of claim 1, wherein the room air and oxygen are delivered via a multi-lumen cannula.

5. The method of claim 1, wherein the oxygen, the room air, or both are delivered in a pulsatile manner.

6. The method of claim 5, wherein the oxygen is delivered continuously, and the room air is delivered in a pulsatile manner.

7. The method of claim 5, wherein the oxygen is delivered in a pulsatile manner, and the room air is delivered continuously.

8. The method of claim 5, wherein the oxygen and the room air are delivered in a pulsatile manner.

9. The method of claim 8, wherein the pulses are delivered at least partially out of phase.

10. The method of claim 1, wherein the oxygen and room air are delivered continuously.

11. The method of claim 5, further comprises the steps of measuring the patient's respiratory rate, and delivering the oxygen and air in pulses synchronized to the patient's respiratory rate.

12. The method of claim 9, wherein the pulses are completely out of phase.

13. The method of claim 12, wherein the oxygen is delivered during inhalation, and room air is delivered during exhalation.
